(19) United States
(12) Patent Application Publication
(10) Pub. No.: US 2010/0227918 A1
(43) Pub. Date: Sep. 9, 2010

(54) STREPTOMYCES-DERIVED ANTIMICROBIAL COMPOUND AND METHOD OF USING SAME AGAINST ANTIBIOTIC-RESISTANT BACTERIA

(75) Inventors: Albert Fliss, Cranbury, NJ (US); Arthur Bailey, Bethel, CT (US); Avraham Yacobi, Englewood, NJ (US)

Correspondence Address: VENABLE LLP, P.O. BOX 34385, WASHINGTON, DC, 20043-9998, US

(73) Assignee: Taro Pharmaceuticals North America, Inc., Grand Cayman (KY)

(21) Appl. No.: 12/293,743

(22) PCT Filed: Apr. 26, 2007
(86) PCT No.: PCT/US07/09974

§ 371 Date: May 5, 2010

Related U.S. Application Data
(60) Provisional application No. 60/794,973, filed on Apr. 26, 2006.

(51) Int. Cl. A61K 31/352; C12N 1/20; C12P 17/18; A01N 43/16; A61P 31/04; A01P 1/00

(52) U.S. Cl. 514/453; 435/253.5; 435/253.6; 435/119

(57) ABSTRACT

The present invention relates to a novel antimicrobial compound of lactoquinomycin that is highly effective against many antibiotic-resistant gram-positive bacteria; namely, methicillin-resistant and vancomycin-resistance Staphylococcus aureus, vancomycin-resistant Enterococcus faecilis and Mycobacteria. The present invention also relates to a fermentation process of culturing a Streptomyces strain to prepare the antimicrobial compound and its use in killing the antibiotic-resistant bacteria.

### FIELD OF THE INVENTION

The present invention relates to an antimicrobial compound highly effective against many antibiotic-resistant gram-positive bacteria; in particular, methicillin-resistant Staphylococcus aureus and vancomycin-resistance Staphylococcus aureus, vancomycin-resistant Enterococcus faecilis and Mycobacteria. The present invention also relates to a fermentation process of culturing a Streptomyces strain to prepare the antimicrobial compound and its use in killing the antibiotic-resistant bacteria.

### BACKGROUND OF THE INVENTION

β-lactam antibiotics such as penicillin was first developed in the 1930s and had once been successful in decreasing morbidity and mortality in microbial infections. (Chopra, I., et al., “The Search for Antimicrobial Agents Effective against Bacteria Resistant to Multiple Antibiotics” Antimicrobial Agents and Chemotherapy, 1997, 41:497-503). It was generally believed in the early 1940s that the threat from infectious diseases was over. Contrary to this common belief, β-lactam resistant bacteria began to become prevalent in the 1950s. Penicillin treatment was no longer effective. New antibiotics having modified β-lactam ring systems (i.e., stable to penicillinase digestion) was introduced in the 1980s (Brown, A. G. “Discovery and Development of New (β-Lactam Antibiotics” Pure & Appl. Chem., 1987, 59:475-484). Yet, the broad use of β-lactam antibiotics has increased the occurrence of antibiotic-resistant microorganisms. These microorganisms include pathogens that cause diarrhea, urinary tract infections, otitis media, meningitis, tuberculosis, gonorrhea, pneumonia, dysentery, wound infections, sinus infections, endocarditis, septicemia, bacteremia and surgical infections. (Lippe, Breakout: The Evolving Threat of Drug-Resistant Diseases, Sierra Clubs, San Francisco, 1995) There is reported an alarming increase in antibiotic-resistant staphylococci, enterococci, streptococci, and pneumococci infections, and a rise in tuberculosis, influenza and sepsis. (“Frontiers in Biotechnology” Science, 1994, 264:359-393).

While 90% of microbial infections are successfully treated with first line antibiotics, over 40% of the infections are resistant to one or more antibiotic (including second line antibiotics). Increasing number of patients in hospitals have become infected with methicillin-resistant Staphylococcus aureus (MRSA), which is becoming a growing health concern. MRSA is distinct from penicillin-resistant S. aureus by virtue of their resistance to all the β-lactam antibiotics such as penicillins, cephalosporins and methicillin, and not merely to penicillin G antibiotics. In antibiotic non-resistant S. aureus, antibiotics kill the bacteria by first binding to bacterial proteins known as “penicillin binding proteins” (PBPs). In MRSA, the PBP (i.e., PBP2′) has shown to have been altered. Antibiotics can no longer bind to PBP2′ and therefore cannot kill the bacteria. In addition, Staphylococcus aureus has the ability to produce the enzyme β-lactamase that can degrade β-lactam. This ability is present in both MRSA and non-MRSA. This enzyme destroys benzyl-penicillin and ampicillin. Other β-lactam antibiotics such as methicillin or cephalothin are resistant to β-lactamase. Various cephem compounds having, at the 7-position, 2-(5-amino-1,2,4-thiadiazol-3-yl)-2(Z)-oxyiminoacetamido group, and having, at the 3-position, pyridiniothiovinyl group, have been reported in JPA S59 (1984)-130292 and JPA H6 (1994)-206886. So far, known cephem compounds are not satisfactory against MRSA.

Microbial infections caused by MRSA are becoming extremely difficult to treat with conventional antibiotics, leading to a sharp rise in clinical complications (Binder, S. et al. Science, 1999, 284:1311. The newest antibiotic, vancomycin, has been shown to be the only antibiotic that is effective against some pathogenic bacteria. It has become the last line of defense against some infections, particularly those by MRSA. However, vancomycin has significant toxicity and is expensive. Broad use of vancomycin has also led to an increased occurrence of vancomycin-resistant Staphylococcus aureus (VRSA) and vancomycin-resistant Enterococcus (VRE). In 1987, it was reported that Enterococcus became resistant to vancomycin. Subsequently in 1996, a clinical isolate of Staphylococcus aureus with reduced susceptibility to vancomycin was reported. This would leave a lack of any reliable treatment for MRSA infection as well as VRSA/VRE infections. Antibiotic-resistant microorganisms are often associated with severe morbidity and mortality among hospitalized patients, particularly among patients with VRE colonizations in long-term care facilities and in those returning to community care, which now present a major public health threat. Management of life-threatening infections caused by antibiotic-resistant strains is particularly difficult, as the range of therapeutic options is very limited.

Antibiotic-resistant bacteria add an estimated $200 million per year to medical costs. When costs of extended hospital stays are considered, the estimated medical costs increase by $30 billion per year. (Phelps, Medical Care, 27: 194-203 (1989))

Over the years, numerous attempts have been made to prepare novel antibiotic other than the known antibiotic. The prepared antibiotic includes structure ranging from simple peptides to complex compounds. For example, U.S. Pat. No. 3,940,479 discloses an antibiotic BN-109 produced by fermentation of genus Bacillus. U.S. Pat. No. 4,294,754 discloses a purified ring peptide antibiotic Permetin A. U.S. Pat. No. 4,536,397 discloses a group of depsipeptide antibiotic, neoviridogriseins I, II and III produced by fermentation of Streptomyces sp. P8648. U.S. Pat. No. 4,759,928 discloses two strains of Streptomyces albovinaceous from soil that produce antibiotic troponemycin against Treponema hyodysenteriae. U.S. Pat. No. 5,939,455 discloses a method of augmenting the therapeutic activity of an oxyalkylene-containing compound with an inhibitor of β-oxidation of fatty acid.

Several attempts have also been made to prepare novel antibiotic against antibiotic-resistant microbial infection. For example, U.S. Pat. No. 6,316,033 discloses a method of using a Chinese herbal composition containing shikonin to treat antibiotic-resistant gram-positive bacteria. U.S. Pat. No. 6,911,525 discloses a lipopeptide compound in the treatment of antibiotic-resistant bacteria. U.S. Pat. No. 6,964,860 discloses a glycopeptide antibiotic isolated from Streptomyces hygroscopicus that has activity against some vancomycin-resistant isolates.

There remains a continuing need in the art for antimicrobial compounds and its use in treating patients with antibiotic-resistant bacteria including MRSA, VRSA, VRE and Mycobacteria.

### SUMMARY OF THE INVENTION

It is an object of the present invention to provide a novel and useful antimicrobial compound effective against antibiotic-resistant gram-positive bacteria such as methicillin-resistant and vancomycin-resistant gram-positive bacteria and mycobacterium. More particularly, the present invention is directed to a purified antimicrobial compound having the formula of:

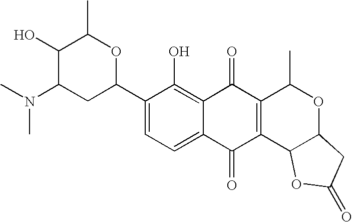

It is another object of the present invention to provide a purified antimicrobial compound of lactoquinomycin.

It is an object of the present invention to provide a pharmaceutical composition containing a purified lactoquinomycin useful in killing antibiotic-resistant gram-positive bacteria including methicillin-resistant (MRSA) and vancomycin-resistance Staphylococcus aureus (VRSA), vancomycin-resistant Enterococcus faecilis (VRE) and Mycobacteria.

It is another object of the present invention to provide a pharmaceutical composition containing a purified antimicrobial compound of lactoquinomycin and its pharmaceutically acceptable salts thereof. The present pharmaceutical composition is useful in treating the antibiotic-resistant bacterial and mycobacterial infections.

It is another object of the present invention to provide a method of treating a bacterial infection in a patient by administering an effective amount of a pharmaceutical composition containing a purified antimicrobial compound of lactoquinomycin. The present pharmaceutical composition is effective in treating antibiotic-resistant bacterial infections including MRSA, VRSA, VRE and Mycobacteria.

It is another object of the present invention to provide to a pharmaceutical composition containing a purified antimicrobial compound of lactoquinomycin used in combination with other antibiotics including β-lactams, aminoglycosides, fluoroquinolones, quinolones, naphthyridines, chloramphenicol, macrolides, ketolides, azalides, tetracyclines, glycopeptides, novobiocin, oxazolidinones and the like. The pharmaceutical composition of the present invention is useful in the therapy of treating infections caused by antibiotic-resistant bacteria and Mycobacteria.

It is another object of the present invention to provide a process of preparation of the antimicrobial compound of lactoquinomycin by fermenting a nutrient medium under aerobic conditions by a Streptomyces species, whereby the lactoquinomycin is recovered and purified from the fermentation broth.

It is another object of the present invention to provide a fermentation process using the Streptomyces strain to prepare lactoquinomycin.

It is another object of the present invention to provide a biological pure culture of Streptomyces species (NRRL B-30919).

It is another object of the present invention to provide a process for recovering antimicrobial compound of lactoquinomycin from a fermentation broth of the Streptomyces strain.

### BRIEF DESCRIPTION OF THE DRAWINGS

FIG. 1 depicts the chemical structure of the antimicrobial compound of lactoquinomycin.

FIG. 2 depicts a diagrammatic representation of “zones of inhibition” in a paper disc assay.

FIG. 3 depicts an antimicrobial activity of various fractions (1-7 fractions) from a flash chromatography.

FIG. 4 depicts an antimicrobial activity of various fractions (1-15 fractions) from a HPLC chromatography.

FIG. 5 depicts a HPLC chromatogram showing a single peak from a purified fraction.

FIG. 6 depicts 1-D ¹H NMR (proton NMR) of the HPLC-purified lactoquinomycin.

FIG. 7 depicts 2-D COSY NMR of the HPLC-purified lactoquinomycin.

FIG. 8 depicts HSQC NMR 2-D of the HPLC-purified lactoquinomycin.

FIG. 9 depicts HMBC NMR (Range: 184-155 PPM) of the purified lactoquinomycin.

FIG. 10 depicts HMBC NMR (Range: 184-155 PPM) of the purified lactoquinomycin.

FIG. 11 depicts HMBC NMR (Range: 184-155 PPM) of the purified lactoquinomycin.

### DETAILED DESCRIPTION OF THE INVENTION

Definitions: As used herein, the term “lactoquinomycin” (also known as medermycin) is intended to encompass lactoquinomycin A and lactoquinomycin B and has a chemical name of 2H-Furo[3,2-b]naphtho[2,3-d]pyran-2,6,11-trione,3,3a,5,11b-tetrahydro-7-hydroxy-5-methyl-8-[2,3,6-trideoxy-3-(dimethylamino)-β-D-arabino-hexopyranosyl]-,(3aR,5R,11bR), or 2H-Furo[3,2-b]naphtho[2,3-d]pyran-2,6,11-trione,3,3a,5,11b-tetrahydro-7-hydroxy-5-methyl-8-[2,3,6-trideoxy-3-(dimethylamino)-β-D-arabino-hexopyranosyl]-,[3aR-(3aα,5α,11bα)], or 2H-Furo(3,2-b)naphtho(2,3-d)pyran-2,6,11-trione,3,3a,5,11b-tetrahydro-7-hydroxy-5-methyl-8-(2,3,6-trideoxy-3-(dimethylamino)-beta-arabino-hexopyranosyl)-, (3aalpha,5alpha,11balpha)-(+); the term “pharmaceutically acceptable” means that which is useful in preparing a pharmaceutical composition that is generally non-toxic and is not biologically undesirable and includes that which is acceptable for human pharmaceutical use; the term “composition” includes, but is not limited to, a powder, a solution, a suspension, a gel, an ointment, an emulsion and/or mixtures thereof; the term composition is intended to encompass a product containing the specified ingredients in the specified amounts, as well as any product, which results, directly or indirectly, from combination of the specified ingredients in the specified amounts; the term “pharmaceutical composition” is intended to encompass a product comprising the active ingredient(s), and a pharmaceutically acceptable excipient; the term “excipient” means a component of a pharmaceutical product that is not the active ingredient, such as filler, diluent, carrier, and so on. The excipients that are useful in preparing a pharmaceutical composition are preferably generally safe, non-toxic and neither biologically nor otherwise undesirable, and are acceptable for human pharmaceutical use. “A pharmaceutically acceptable excipient” as used in the specification and claims includes both one and more than one such excipient; the term “treating” refers to treating, preventing or ameliorating the symptoms of microbial infection; the term “effective amounts” refers to an amount, when administered for treating or prevent a disease, is sufficient for effect of treating, preventing, or ameliorating microbial infection; the term“effective amount” will vary depending on the compound, the disease and its severity and the age, weight, etc. of the patient to be treated; the term “purified” compound refers to a compound having a purity of at least 90%, it encompasses a purity of 95% or 99%; the term “antibiotic” refers to a chemical compound produced by one microorganism that inhibits the growth of or kill a different microorganism; “antibiotic-resistant” refers to a microorganism whose growth cannot be killed by commonly used antibiotic, such as ampicillin, ceftriaxone, erythromycin, morfloxacin streptomycin, sulfisoxazole, and tetracycline; “MRSA” refers to methicillin-resistant Staphylococcus aureus; “VRSA” refers to vancomycin-resistant Staphylococcus aureus; “VRE” refers to vanomycin-resistant Enterococcus; “Mycobacteria” refers to an unencapsulated, strongly acid-fast rod that frequently shows irregular beading due to vacuoles and polyphosphate granules; “Enterococcus” refers to a gram-positive coccus; “Staphylococcus” refers to a genus in the Micrococcaceae family and is classified as a gram-positive cocci and divided into two major groups: aureus and non-aureus; “fermentation” broadly refers broadly to the bulk growth of microorganisms on a growth medium. No distinction is made between aerobic and anaerobic metabolism; “zone of inhibition” refers to a clear ring appearing around a paper disc containing an antibiotic. If the antibiotic works successful, there is a “zone of inhibition.” The larger the “zone of inhibition”, the more effective that antibiotic is against that particular type of microorganism; “microbial colony” refers to the progeny of a single microbial cell in the original inoculum.

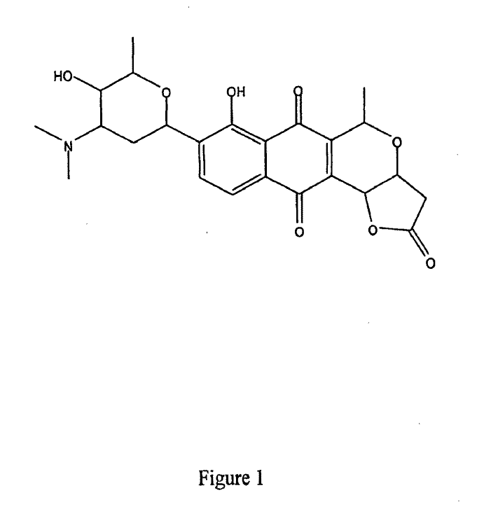

The present invention relates to a novel antimicrobial compound having a structural formula as depicted in FIG. 1. The present antimicrobial compound is lactoquinomycin.

In one embodiment, the present invention provides a pharmaceutical formulation containing the novel antimicrobial compound of lactoquinomycin. The present pharmaceutical formulation contains the antimicrobial compound having a structural formula as depicted in FIG. 1 and is called lactoquinomycin.

In another embodiment, the present invention provides a pharmaceutical formulation comprising the antimicrobial compound having a structural formula as depicted in FIG. 1 and is called lactoquinomycin, and a physiologically acceptable salt thereof. Salts of the antimicrobial compound of the present invention include salts of the disclosed compound that are modified by making acid or base salts. In another embodiment, the salt is a pharmaceutical acceptable salt, which embraces salts commonly used to form alkali metal salts and to form addition salts of free acids or free bases. Examples of pharmaceutical acceptable salts includes, but not limited to, sodium, potassium, calcium and the like.

The pharmaceutically acceptable salts referred to above also include addition salts in the form of salts derived from inorganic or organic alkali. Included among such salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate and undecanoate. These salts may be obtained by employing conventional procedures such as, for example, by mixing solutions containing equimolar amounts of the free acid and the desired alkali together, followed by filtration to collect the required salt, if insoluble, or else by evaporation of the solvent form the system in accordance with standard techniques.

In another embodiment, the present invention provides a pharmaceutical formulation further comprising one or more pharmaceutically acceptable carriers. The carrier(s) must be acceptable as being compatible with the ingredients of the formulation and not deleterious to the recipients thereof. The present pharmaceutical formulation may conveniently be presented as a pharmaceutical formulation in unit dosage form.

Pharmaceutical formulations include those suitable for oral, topical (including dermal, buccal and sublingual), rectal, parenteral (including subcutaneous, intradermal, intramuscular and intravenous), nasal and pulmonary administration e.g. by inhalation. The formulation may, where appropriate, be conveniently presented in discrete dosage units and may be prepared by any of the methods well known in the art of pharmacy. All methods include the step of bringing into association the present antimicrobial compound or a physiologically acceptable salt thereof with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of the active ingredient. A tablet may be made by compression or moulding, optionally with one or more accessory ingredients. Compressed tablets may be prepared by compressing in a suitable machine the active ingredient in a free-flowing form such as a powder or granules optionally mixed with a binder, lubricant, inert diluent, lubricating agent, surface-active agent or dispersing agent. Moulded tablets may be made by moulding an inert liquid diluent. Tablets may be optionally coated and, if uncoated, may optionally be scored. Capsules may be prepared by filling the active ingredient, either alone or in admixture with one or more accessory ingredients, into the capsule shells and then sealing them in the usual manner. Cachets are analogous to capsules wherein the active ingredient together with any accessory ingredient(s) is sealed in a rice paper envelope. Granules may be packaged e.g. in a sachet. Formulations suitable for oral administration wherein the carrier is a liquid may be presented as a solution or a suspension in an aqueous liquid or a non-aqueous liquid, or as an oil-in-water liquid emulsion.

Pharmaceutical formulations suitable for parenteral administration include sterile solutions or suspensions of the active ingredient in aqueous or oleaginous vehicles. Injectable preparations may be adapted for bolus injection or continuous infusion. Such preparations are conveniently presented in unit dose or multi-dose containers which are sealed after introduction of the formulation until required for use. Alternatively, the active ingredient may be in powder form, which is constituted with a suitable vehicle, such as sterile, pyrogen-free water, before use.

In another embodiment, the present invention provides the active ingredient may be in the form of a solution or suspension for use in an atomiser or nebuliser whereby an accelerated airstream or ultrasonic agitation is employed to produce a fine droplet mist for inhalation. Such solutions or suspensions may comprise, in addition to the active ingredient and solvent(s), optional ingredients such as surfactants. Suitable surfactants include those described above for self-propelling formulations. When a suspension of the active ingredient is employed, the compound is preferably in finely divided form, e.g. in micronized form.

Formulations suitable for nasal administration include presentations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve.

It should be understood that in addition to the aforementioned carrier ingredients the pharmaceutical formulations for the various routes of administration described above may include, as appropriate one or more additional carrier ingredients such as diluents, buffers, flavoring agents, binders, surface active agents, thickeners, lubricants, preservatives (including anti-oxidants) and the like, and substances included for the purpose of rendering the formulation isotonic with the blood of the intended recipient.

In another embodiment, the present pharmaceutical formulation may optionally include other therapeutic and/or prophylactic ingredients may be included. For example, the present invention provides an antimicrobial compound of lactoquinomycin in combination with a known antibiotic. These antibiotics include, but not limited to, cephalosporins, penicillins, gentamicin, ciprofloxacin, chloramphenicol, vancomycin, teicoplainin, and the like.

The pharmaceutical composition of the present invention is effective against enterococcal infections, particularly against clinical isolates of multiply resistant E. faecium. The pharmaceutical composition is effective against many gram-positive bacteria including, but not limited to, MRSA, VRSA, VRE, and Mycobacteria.

Preferably, the pharmaceutical composition of the present invention is suitable for the treatment of microbial infection diseases caused by antibiotic-resistant gram-positive bacteria. This includes, but not limited to, microbial infection diseases caused by MRSA, VRSA, VRE and the like. More preferably, the microbial infection diseases caused by MRSA and VRSA include bacteremia, pneumonia, osteomyelitis, cellulitis, abscesses, endocarditis and the like. More preferably, the microbial infection diseases caused by VRE include bacteremia, pneumonia, osteomyelitis, cellulitis, abscesses, endocarditis, urinary tract infection and the like. The present pharmaceutical composition is also suitable against clinical isolates of multiply resistant E. faecium.

Preferably, the pharmaceutical composition of the present invention is suitable for the treatment of microbial infection diseases caused by antibiotic-resistant gram-positive bacteria. This includes, but not limited to, microbial infection diseases caused by MRSA, VRSA, VRE and the like. More preferably, the microbial infection diseases caused by MRSA and VRSA include bacteremia, pneumonia, osteomyelitis, cellulitis, abscesses, endocarditis and the like. More preferably, the microbial infection diseases caused by VRE include bacteremia, pneumonia, osteomyelitis, cellulitis, abscesses, endocarditis, urinary tract infection and the like. The present pharmaceutical composition is also suitable against clinical isolates of multiply resistant E. faecium.

Preferably, the pharmaceutical composition of the present invention is suitable for the treatment of microbial infection diseases caused by acid-fast mycobacterium. This includes, but not limited to, microbial infection diseases caused by Mycobacterium tuberculosis, Mycobacterium leprae, Mycobacterium avium complex, Mycobacterium avium subspecies paratuberculosis, Mycobacterium palustre, Mycobacterium phlei, Mycobacterium smegmatis, and the like. More preferably, tuberculosis caused by Mycobacterium tuberculosis and the like. More preferably, leprosy caused by Mycobacterium leprae and the like. More preferably, disseminated disease in AIDS caused by Mycobacterial avium Complex and the like. More preferably, Crohn's disease caused by Mycobacterium avium subspecies paratuberculosis and the like.

Suitable subjects for the administration of the pharmaceutical formulation of the present invention include mammals, primates, man, and other animals. In vitro antimicrobial activity is predictive of in vivo activity when the compositions are administered to a mammal infected with a susceptible bacterial organism.

In one embodiment, the present invention provides a fermentation process for preparing the antimicrobial compound of lactoquinomycin. In accordance with the present invention, the cultures were performed in a cultured medium that comprise a carbon source and a nitrogen source. The carbon source and the nitrogen source and additional nutritional requirements may be conveniently determined by one skilled in the art.

The carbon source used in the described experiments for microbial colony no. 59 was starch (see Examples 4 and 5). Illustrative examples of other suitable supplemental carbon sources include, but are not limited to, other carbohydrates, such as glucose, fructose, mannitol, starch or starch hydrolysate, cellulose hydrolysate and molasses; organic acids, such as acetic acid, propionic acid, lactic acid, formic acid, malic acid, citric acid, and fumaric acid; and alcohols, such as glycerol, inositol, mannitol and sorbitol.

The nitrogen source used in the described experiments for microbial colony no. 59 was yeast extract. Illustrative examples of suitable nitrogen sources include, but are not limited to, ammonia, including ammonia gas and aqueous ammonia; ammonium salts of inorganic or organic acids, such as ammonium chloride, ammonium nitrate, ammonium phosphate, ammonium sulfate and ammonium acetate; urea; nitrate or nitrite salts, and other nitrogen-containing materials, including amino acids as either pure or crude preparations, meat extract, peptone, fish meal, fish hydrolysate, corn steep liquor, casein hydrolysate, soybean cake hydrolysate, yeast extract, dried yeast, ethanol-yeast distillate, soybean flour, cottonseed meal, and the like.

Besides the carbon and nitrogen sources, the culture medium contains suitable inorganic salts, and, as appropriate, various trace nutrients, growth factors and the like suitable for cultivation of the microorganism strain. Additional nutrients used in the described experiments for microbial colony no. 59 were dibasic sodium phosphate, and magnesium sulfate. Illustrative examples of suitable inorganic salts include, but are not limited to, salts of potassium, calcium, sodium, magnesium, manganese, iron, cobalt, zinc, copper, molybdenum, tungsten and other trace elements, and phosphoric acid.

Illustrative examples of appropriate trace nutrients, growth factors, and the like include, but are not limited to, coenzyme A, pantothenic acid, pyridoxine-HCl, biotin, thiamine, riboflavin, flavine mononucleotide, flavine adenine dinucleotide, DL-6,8-thioctic acid, folic acid, Vitamin B₁₂, other vitamins, amino acids such as cysteine and hydroxyproline, bases such as adenine, uracil, guanine, thymine and cytosine, sodium thiosulfate, p- or r-aminobenzoic acid, niacinamide, nitriloacetate, and the like, either as pure or partially purified chemical compounds or as present in natural materials.

The amount of each of these ingredients to be employed is preferably selected to maximize the production of antimicrobial agent. Such amounts may be determined empirically by one skilled in the art according to the various methods and techniques known in the art.

The fermentation culture conditions employed, including temperature, pH, aeration rate, agitation rate, culture duration, and the like, may be determined empirically by one of skill in the art to maximize the production. The selection of specific culture conditions depends upon factors such as the medium composition and type, culture technique, and similar considerations. In a preferred embodiment of the present invention, cultivation takes place at a temperature in the range of about 26° C. to 40° C., preferably at a temperature of about 37° C.; and at a pH in the range of 5 to 9, preferably in the range of 6.5 to 7.5. The culture conditions employed can, of course, be varied by known methods at different time-points during cultivation, as appropriate, to maximize production of the antimicrobial compound. Preferably, the time of fermentation is about 3 days to about 12 days. Preferably, the time of fermentation is about 7 days.

Fermentation culture of the microorganism strain may be accomplished using any of the submerged fermentation techniques known to those skilled in the art, such as airlift, traditional sparged-agitated designs, or in shaking culture.

The following Examples are provided for illustrative purposes only, it is to be understood that both the foregoing description and the detailed description are not intended to limit the scope of the present invention.

### EXAMPLES

The experimental scheme is provided in details hereinafter for illustrating the isolation and purification of an antimicrobial compound.

i) Soil Sample Collection and Preparation

Soil samples in Westchester County and Putnam County of New York were collected. Specifically, soil samples under decayed leaf beds were collected in order to maximize the chances of obtaining Streptomyces and Actinomycetes species. Caution was exercised to preclude leaves and extraneous matters. Soil samples were collected into sterile 50-mL polypropylene centrifuge tubes. Upon return to the laboratory, 0.5 gram of each soil sample was weighed. Soil samples were subsequently diluted with 9.5 mL sterile distilled water and mixed by vortex. Ten (10) μl of the vortexed soil samples were diluted with 10 ml, sterile distilled water to make a 1,000 fold dilution of the soil samples.

ii) Functional Screening For Microbial Isolates Having Antimicrobial Activity (Primary Screening)

A primary screening was performed to identify microorganism isolates present in the soil samples that have antimicrobial activity. The primary screening permits the identification of potential isolates that possess antimicrobial activity. 100 μl of the diluted soil samples were plated onto the following agar plates:

1) Actinomycetes Agar;

2) ISP2 Agar;

3) Brain Heart Agar;

4) Emerson Agar;

5) Mineral Agar;

6) Soil (HB) Agar;

7) Humic Acid Agar;

8) Bennett's Agar;

9) Tryptone Soy Agar;

10) Yeast Malt Agar;

11) Czapek Agar; and

12) Starch Casien Agar.

Plates were incubated at 30° C. for approximately two (2) weeks after being spread with sterile glass beads for uniform distribution. Growth of microbial colonies was observed over the same two-week period. Observation was made as to the interaction between different microbial colonies on the plates. A microbial colony that inhibits the growth of a neighboring microbial colony (i.e., creating a “zone of inhibition” around itself) is an indication for the presence of an antimicrobial activity generated by the microbial colony.

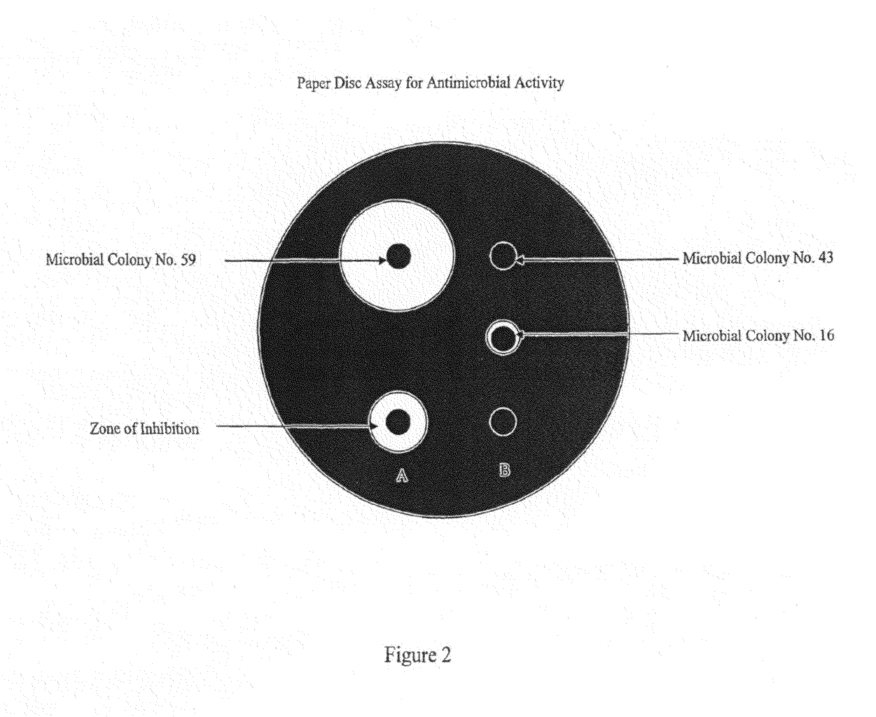

As depicted diagrammatically in FIG. 2, a microbial colony (colony no. 59) was identified that exhibited a zone of inhibition (++++++) (see, page 20, lines 29-30 and page 21, lines 1-2) when cultured in Emerson agar plate (see above). Microbial colony #16 also exhibited a zone of inhibition (+) when cultured. In contrast, microbial colony no. 43 did not exhibit a zone of inhibition.

Microbial colonies that exhibited a zone of inhibition were isolated. Individual microbial colonies that formed zones of inhibition were streaked for isolation using standard microbiological techniques (See, Molecular Cloning, A Laboratory Manual). Using this approach, approximately one hundred thousand (100,000) microbial colonies had been tested. One hundred and twenty-two (122) microbial colonies were tested positive (i.e., exhibited a zone of inhibition). The 122 microbial colonies were isolated in the initial screening.

Each of the 122 microbial colonies was further isolated by successive streaking and characterized as follow. Successive streaking was performed until a single microbial colony based on morphology was observed. Stocks of each single microbial colony were grown in the liquid version of the agar (0.4% yeast extract, 1.5% starch, 0.1% Na₂HPO₄, and 0.01% MgSO₄; pH 6.8) in preparation of a secondary screening.

iii) Functional Screening For Microbial Isolates Having Antimicrobial Activity (Secondary Screening)

While the primary screening was to identify the presence of any potential antimicrobial activity, the secondary screening was focused to determine the spectrum of its antimicrobial activity.

Liquid cultures of the stocks as prepared from the initial screening were used. Specifically, each of the single microbial colonies from the stocks was inoculated into two (2) mL of liquid broth. Liquid cultures prepared from the microbial stocks were incubated at 30° C. for a time period of two (2) weeks.

Samples (100 μl) from the liquid cultures were harvested daily for a 2-week culture period. The harvested samples were centrifuged at 13,000 rpm for 2 minutes to remove cells. Supernatants were transferred to microcentrifuge tubes, and were concentrated (−10-fold) using a SpeedVacuum. The concentrated supernatants were stored at 4° C. and the spectrum of the antimicrobial activities was determined.

Spectrum of Antimicrobial Activity: Spectrum of antimicrobial activity of the concentrated supernatants was determined using the following antimicrobial assay. Specifically, ten (10) μl of the concentrated supernatants were assayed against the following four (4) microorganisms; namely,

1) Micrococcus haeus (gram-positive bacteria);

2) E. coli (gram-negative bacteria);

3) Candida albicans (yeast/fungi); and

4) Pseudomonas aeriginosa (gram-negative bacteria).

The antimicrobial assay was performed as described in USP (See, Molecular Cloning, A Laboratory Manual). In brief, tested microorganism strains were grown in optimal culture media for growth at the optimal growth temperature for about 16 hours with shaking. Tested plates were prepared as follows. First, base agar layer-media were prepared as per USP. The media were then autoclaved, and cooled to 48° C. Once cooled to 48° C., 20 mL of the cooled media was poured onto a sterile polystyrene petri dish. This base agar layer was further allowed to solidify for 30 minutes (agar was added as a solidifying agent). Second, seed agar layers were prepared as per USP. The media were then autoclaved, and cooled to 48° C. Once cooled to 48° C., 1 mL of tested microorganism strain (already grown for 16 hours as described in the first step above) was added to the seed agar media. The seed agar media was gently swirled to mix. Five (5) mL of the seed agar media was pipetted on top of the base agar layer. Care was taken to avoid air bubbles. This seed agar media was allowed to cool for about 15 minutes at room temperature. Plates were stored at 4° C. until needed.

Ten (10) μl of each of the concentrated supernatants (described above) were pipetted onto a sterile filter paper disc. The sterile filter paper discs were dried in air for approximately 5 minutes. The sterile filter paper discs were carefully applied onto the surface of the plates (prepared as described above). Plates holding sterile filter discs were incubated either at 37° C. (for Micrococcus luteus, E. coli, and Pseudomonas aeriginosa) or at 30° C. (for Candida albicans) for a timer period of 24 hours. Negative controls included liquid culture media. Positive controls included known antimicrobial compounds such as vancomycin (against gram-positive bacteria), tetracycline (against gram-positive/gram-negative bacteria), and amphotericin B (against yeast/fungi).

At the end of the 24-hour incubation, plates were visualized and checked for the presence of any zones of inhibition exhibited by concentrated supernatants from respective microbial colonies. A zone of inhibition having a diameter of greater than 7 mm (the diameter of a sterile filter paper disc is 5 mm) was treated as positive (i.e., presence of antimicrobial activity). Invariably, negative controls did not exhibit any zone of inhibition; while all positive controls (i.e., vancomycin, tetracycline and amphotericin B) exhibited a zone of inhibition greater than 7 mm (e.g., 20-25 mm in general).

Using this approach, the 122 microbial colonies isolated during the initial screening were tested. As shown in the following Table 1, out of the 122 microbial colonies, six (6) microbial colonies were identified and characterized in the secondary screening:
1) five (5) of the six (6) microbial colonies were found to possess antimicrobial activity against Micrococcus luteus;
2) two (2) of the six (6) microbial colonies were found to possess antimicrobial activity against E. coli;
3) two (2) of the six (6) microbial colonies were found to possess antimicrobial activity against Pseudomonas aeriginosa; and
4) two (2) of the six (6) microbial colonies were found to possess antimicrobial activity against Candida albicans.

TABLE 1
Antimicrobial Spectrum of Activity For the Six (6)
Microbial Colonies After The Secondary Screening

| | Number of Positive | Microbial Colony |
|---|---|---|
| Tested Microorganisms | Soil Isolates | (Colony Number) |
| Gram (−) Bacteria | | |
| E. coli | 2 | 24, 37 |
| Pseudomonas aeriginosa | 2 | 24, 37 |
| Gram (+) Bacteria | | |
| Micrococcus luteus | 5 | 16, 24, 25, 37, 59 |
| Fungus | | |
| Candida albicans | 2 | 24, 26 |

iv) Functional Screening For Microbial Isolates Having Antimicrobial Activity (Tertiary Screening)

While the secondary screening was to identify the broad spectrum of antimicrobial activities for the microbial isolates, the tertiary screening was focused to determine the specific spectrum of the antimicrobial activities; All six (6) microbial colonies tested positive in the secondary screening were subjected to tertiary screening.

Microbial colony no. 59 was shown to have antimicrobial activity against Micrococcus luteus in the secondary screening. Presented herein this application is the result of only one (1) microbial colony (i.e., colony no. 59). The microbial colony no. 59 was chosen to undergo a tertiary screening for antimicrobial activity against twelve (12) other microorganisms, including: 1) Staphylococcus aureus; 2) methicillin-resistant Staphylococcus aureus; 3) vancomycin-resistant Staphylococcus aureus; 4) Enterococcus faecilis; 5) vancomycin-resistant Enterococcus faecilis; 6) Mycobacteria palustre; 7) Mycobacteria phlei; 8) Mycobacterium smegmatis; 9) E. coli; 10) Pseudomonas aeriginosa; 11) Serratia marcesens; and 12) Candida albicans.

Antimicrobial Assays: 10 μl of the 10-fold concentrated supernatants from each of the six (6) isolated colony was assayed for their antimicrobial activity against the twelve (12) microorganisms as listed above. The antimicrobial assay used in the tertiary screening was similar to that used in the secondary screening. In brief, tested microorganism strains were grown in optimal culture media for growth at the optimal growth temperature for about 16 hours with shaking. Tested plates were prepared as follows. First, base agar layer-media were prepared as per USP. The media were then autoclaved, and cooled to 48° C. Once cooled to 48° C., 20 mL of the cooled media was poured onto a sterile polystyrene petri dish. This base agar layer was further allowed to solidify for 30 minutes (agar was added as a solidifying agent). Second, seed agar layers were prepared as per USP. The media were then autoclaved, and cooled to 48° C. Once cooled to 48° C., 1 mL of tested microorganism strain (already grown for 16 hours as described in the first step above) was added to the seed agar media. The seed agar media was gently swirled to mix. Five (5) mL of the seed agar media was pipetted on top of the base agar layer. Care was taken to avoid air bubbles. This seed agar media was allowed to cool for about 15 minutes at room temperature. Plates were stored at 4° C. until needed.

Ten (10) μl of each of the concentrated supernatants were pipetted onto a sterile filter paper disc. The sterile filter paper discs were dried in air for approximately 5 minutes. The sterile filter paper discs were carefully applied onto the surface of the plates. Plates holding sterile filter discs were incubated either at 37° C. (for Staphylococcus aureus, MRSA, VRSA, Enterococcus faecilis, VRE, Micrococcus luteus, E. coli, Serratia marcesens and Pseudomonas aeriginosa) or at 30° C. (for Candida albicans) for a time period of 24 hours. Negative controls included liquid culture media. Positive controls included known antimicrobial compounds such as vancomycin (against gram-positive bacteria), tetracycline (against gram-positive/gram-negative bacteria), and amphotericin B (against yeast/fungi).

At the end of the 24-hour incubation, plates were visualized and checked for the presence of any zones of inhibition exhibited by concentrated supernatants from respective microbial colonies. A zone of inhibition having a diameter of greater than 7 mm (the diameter of a sterile filter paper disc is 5 mm) was treated as positive (i.e., presence of antimicrobial activity). Invariably, negative controls did not exhibit any zone of inhibition; while all positive controls (i.e., vancomycin, tetracycline and amphotericin B) exhibited a zone of inhibition greater than 7 mm (e.g., 20-25 mm in general).

In general, a zone of inhibition visualized as having a diameter of about 7-10 mm is classified as “+”; a diameter of about 11-14 mm is “++”; a diameter of about 15-18 mm is “+++”; a diameter of about 19-22 mm is “++++”, and a diameter of greater than 23 mm is classified as “+++++”.

As shown in the Table 2, microbial colony no. 59 exhibited very strong antimicrobial activity against the tested gram-positive bacteria including Staphylococcus aureus, Enterococcus, and Mycobacteria. Notably, the microbial colony 59 did not exhibit any antimicrobial effect against the tested gram-negative bacteria and yeast/fungus, indicating specificity of its antimicrobial activity.

TABLE 2
Antimicrobial Spectrum of Activity for Microbial Colony No. 59

| | Antimicrobial Activity For | Known Antibiotic |
|---|---|---|
| Tested Microorganisms | Microbial Colony No. 59 | Controls |
| Gram (+) Bacteria | | |
| Staphylococcus aureus | +++++ | vancomycin (++) |
| Methicillin-Resistant Staphylococcus aureus | +++++ | vancomycin (++) |
| Vancomycin-Resistant Staphylococcus aureus | +++++ | (−)* |
| Enterococcus faecilis | ++++ | vancomycin (++) |
| Vancomycin-Resistant Enterococcus faecilis | ++++ | (−)* |
| Mycobacterium palustre | +++++ | vancomycin (++++) |
| Mycobacterium phlei | +++++ | vancomycin (++++) |
| Mycobacterium smegmatis | +++++ | vancomycin (++++) |
| Gram (−) Bacteria | | |
| E. coli | − | tetracycline (+++++) |
| Pseudomonas aeriginosa | − | gentamicin (+++++) |
| Serratia marcesens | − | gentamicin (+++++) |
| Fungi | | |
| Candida albicans | − | amphotericin B (+++++) |

“−” refers to “no detectable microbial activity”;
“+” refers to “weak antimicrobial activity” relative to that of microbial colony no. 59;
“+++” refers to “strong antimicrobial activity” relative to that of microbial colony no. 59 and
“+++++” refers to “very strong antimicrobial activity” relative to that of microbial colony no. 59; and
“*” refers to “no commercial antibiotic testing disc available”

### Characterization of Microbial Colony No. 59

Color Characteristics Aerial growth of microbial colony no. 59 exhibited a rough leathery appearance when plated on Emerson agar media plates. The appearance of the isolated microbial colonies demonstrated jagged crusty edges, a leathery textured, slightly raised colony appearance with an irregular shape. These colonies were brown and/or grayish brown in appearance with multiple shades in a single colony.

Growth and Production Conditions: Based on its growth on Emerson agar, colony morphology, color and the presence of Geosmin in the culture media the microorganism that produces the antibiotic compound (accounting for its antimicrobial activity) was presumed to be of the genus Streptomyces.

This microorganism was found to grow in the temperature range of about 26° C. to about 37° C. on Emerson media/agar. Microbial colony no. 59 was found to produce the antibiotic compound at varying levels depending on the incubation temperatures. Typically the brown color and antibiotic production emerge at a similar time point ranging from about 2-4 days when grown at the 2-liter size. When grown at the 2-ml size, the brown color and antibiotic activity emerged at about 1-2 days. This was likely due to the amount of inoculate when comparing the 2-ml to 2-liter sizes. A single colony was sub-cultured and stored on plates. Viability and ability to produce antibiotic were maintained at least for the tested time and temperature periods (i.e., several months at room temperature and/or 4° C.).

Ribosomal (16S) DNA Sequence

To determine if microbial colony no. 59 was a novel species of Streptomyces, ribosomal DNA (rDNA) was amplified and isolated using standard PCR technique (See, Molecular Cloning, A Laboratory Manual). The PCR fragment (˜500 bp size) was subcloned directly into PGEM T-easy using standard Molecular Biological method (See, Molecular Cloning, A Laboratory Manual). The nucleotide sequence of the rDNA was sequenced and was compared using BLAST analysis to all known 16 S rDNA sequences of Streptomyces species.

Ribosomal (16S) sequencing revealed various degree of homology at the nucleotide sequences (spanning 421 by fragment). In particular, the highest degree of homology was exhibited between the nucleotide sequence of the 16 S of microbial colony no. 59 and that of Streptomyces bikiniensis strain DSM 40581.

It is therefore concluded that microbial colony no. 59 belong to the Streptomyces genus and represents a novel Streptomyces species having a 96% homology (rDNA) to its mostly related species.

Microorganism Deposit

A subculture of the microbial colony no. 59 has been deposited (NRRL 8-30919) in connection with the present invention. The present microbial strain, prepared and used for carrying out the examples, has been deposited at the Agricultural Research Service Culture Collection (NRRL), located at 1815 North University Street, Peoria, Ill. 61604 U.S.A., pursuant to the Budapest Treaty for the International Recognition of the Deposit of Microorganisms for the Purpose of Patent Procedure. All restrictions on the availability of the materials deposited will be irrevocably removed upon the issuance of a patent thereon. The microorganism deposit was a Streptomyces species.

Fermentation of Streptomyces SPECIES for Microbial Colony

Two (2) suitable fermentation methods were used to culture the Streptomyces species of microbial colony no. 59. The 2 fermentation methods were as follows: i) fermentation in a fermentor, and ii) fermentation in a shake-flask.

i) Fermentation in a Fermentor for Microbial Colony No. 59

Initial cultures of microbial colony no. 59 were inoculated from a single microbial colony of the stock (Emerson Agar plate) containing the microbial colony no. 59. The colonies were inoculated into 2 mL of liquid Emerson media (yeast extract 4 g/L, starch 15 g/L, Na₂HPO₄ 1 g/L and MgSO₄ 0.5 g/L, pH 6.8). Incubation of the colonies in the liquid Emerson media was conducted under shaking condition (250 rpm) at 26° C. for a time period of 48 hours or until brown pigment appeared. The liquid culture was diluted 100-fold with 200 mL of fresh liquid Emerson media. Incubation of the colonies in the diluted liquid culture was conducted under shaking condition (250 rpm) at 26° C. for an addition time period of 8 hours.

The 200 mL liquid Emerson media (containing the microbial colony no. 59) was added into a fermentor (3.3 L New Brunswick Scientific BioFlo III fermentor) that contained 2 liters of freshly-prepared liquid Emerson media. The 2.2 L of liquid culture media was incubated for a time period of 7 days. Incubation of the colonies in the fermentor was conducted under stirring condition (500 rpm) at 26° C. and aerated at 3.3 L air per minute. Aliquots of 10 mL liquid culture were sampled from various time points during the fermentor fermentation (i.e., days 2, 3, 4, 5, 6, and 7). The antimicrobial activity present in the aliquots of the liquid culture from various time points was determined against MRSA using the antimicrobial assay as described above. Initial studies indicated that the antimicrobial activity of the liquid culture (for microbial colony no. 59) began to appear at day 2, and reached its maximal level at day 5.

ii) Fermentation in a Shake-Flask for Microbial Colony No. 59

Initial cultures of microbial colony no. 59 were inoculated from a single microbial colony of the stock (Emerson Agar plate) containing the microbial colony no. 59. The colonies were inoculated into 2 mL of liquid Emerson media. Incubation of the colonies in the liquid culture was conducted under shaking condition (250 rpm) at 26° C. for a time period of 48 hours or until brown pigment appeared. The liquid culture was diluted 100-fold with 200 mL of fresh liquid Emerson media. Incubation of the colonies in the diluted liquid culture was conducted under shaking condition (250 rpm) at 26° C. for an additional time period of 8 hours.

The 200 mL liquid culture Emerson media was added to a 4 L Erlenmeyer flask containing 2 L of freshly-prepared liquid Emerson media. Incubation of the colonies in the shake flask was conducted under shaking condition (250 rpm) at 26° C. Aliquots of 10 mL liquid culture were sampled from various time points during the shake flask fermentation (i.e., days 3, 4, 5, 6, 7, 8 and 9). The antimicrobial activity present in the aliquots of the liquid culture from various time points was determined against MRSA using the antimicrobial assay as described above. Initial studies indicated that the antimicrobial activity of the liquid culture (for microbial colony no. 59) began to appear at day 3, and reached its maximal level at day 9.

Preparation of Fermentation Broth

At the end of the fermentation, fermentation broth was prepared as follow. The fermentation media containing the microbial colony and the associated antimicrobial activity were transferred to 250 mL centrifuge bottles. Centrifugation of the 250 mL bottles was performed at 6,000×g for 10 minutes at 4° C. The centrifugation was sufficient to remove the microorganisms from the fermentation broth. The fermentation broth was obtained by simply decanting the supernatant. The antimicrobial activity was shown to be present in the fermentation broth. Initial studies reveal that the antimicrobial activity was stable in the fermentation broth for at least 6 months when stored at 4° C.

Purification of Antimicrobial Compounds from Fermentation Broth

Purification of antimicrobial compound(s) present in a fermentation broth (from the fermentation culture of microbial colony no. 59) was performed using three (3) separation steps; namely, i) extraction using a hydrophobic resin, ii) separation using a flash column chromatography, and iii) purification using a HPLC chromatography. The three (3) separation steps were shown to purify an antimicrobial compound to its homogeneity greater than 99% (i.e., >99% purity). The purified compound was then subjected to NMR analysis for chemical structure delineation.

A fermentation broth (˜750 L) was prepared using the procedure as described above after the fermentation culture of microbial colony no. 59. Aliquots of fermentation broth (i.e., 20 L) were added to a container followed by the extraction step using a hydrophobic resin. The eluates were then combined to form a volume of ˜200 mL prior to the separation step using a flash column chromatography.

i) Extraction Step Using a Hydrophobic Resin

A hydrophobic resin (i.e., Diaion SP-207 resin) was used to extract antimicrobial compound(s) from fermentation broth. The hydrophobic resin was first prepared by reconstituting it in methanol (i.e., 200 grams of resin was reconstituted into 1 L of 100% methanol) for about 1 hour. Methanol was removed by filtration through a Buchner funnel. The hydrophobic resin was washed thoroughly with distilled water (3× with 1 L each). The hydrophobic resin was then resuspended in a minimum amount of water (i.e., ˜200 mL).

Without being bound by a theory, it is believed that a hydrophobic resin functions to bind to a hydrophobic region of a compound so as to retain onto the hydrophobic resin. It is further believed that the present antimicrobial compound can effectively bound onto a hydrophobic resin. For example, about 90% of the antimicrobial activity present in the fermentation broth was retained onto the hydrophobic resin after incubation of the fermentation broth for 3 hours with the Diaion SP-207 resin. Suitable hydrophobic resins may be used to retain the present antimicrobial compound via a hydrophobic-hydrophobic interaction. Exemplified hydrophobic resins include, but not limited to, Diaion HP series, Diaion SP series and XAD series.

Two hundred grams (200 grams) of the prepared hydrophobic resin was added into 20 L of the fermentation broth. Hydrophobic resin was allowed to incubate with the fermentation broth for 3 hours at room temperature. The mixture was under mechanical stirring (250 rpm) to form a resin slurry. This allows the antimicrobial compound(s) to bind to the hydrophobic resin.

At the end of the 3-hour incubation, the mixture containing fermentation broth and hydrophobic resin was filtered through a Buchner funnel to remove the liquid component of the mixture. The obtained hydrophobic resin was washed with 1 L of distilled water. The washing was repeated (2×) to remove both the unbound and the nonspecific bound materials from the hydrophobic resin.

Antimicrobial compound(s) were extracted from the fermentation broth and bound onto the hydrophobic resin. The bound antimicrobial compound(s) were eluted from the hydrophobic resin. The hydrophobic resin was mixed with 200 mL of a solution containing 100% acetonitrile and 0.05% trifluoroacetic acid. The mixture was incubated for 30 minutes at room temperature. The mixture was shaken (250 rpm) during the 30 minute-incubation time period. This allows the antimicrobial compound(s) to be eluted from the hydrophobic resin. The mixture containing the eluted antimicrobial compound(s) and the hydrophobic resin was filtered through a Buchner funnel to remove the hydrophobic resin.

Hydrophobic resin was further eluted (2×) using 200 mL of a solution containing 100% acetonitrile and 0.05% trifluoroacetic acid and filtered. All resin eluates were combined and pooled together into a glass container. Resin eluates were concentrated to 10-fold to remove acetonitrile and trifluoroacetic acid using a rotary evaporator. The concentrated resin eluates were stored at 4° C.

Antimicrobial activity of the fermentation broth (before and after extraction with the hydrophobic resin) was determined against MRSA using the antimicrobial assay as described above. Before the extraction, there was a strong antimicrobial activity present in the fermentation broth. Our data indicated that greater than 99% of the antimicrobial activity was recovered in the resin eluates, and only negligible amount of antimicrobial activity was found to remain in the fermentation broth (that was after the extraction with the hydrophobic resin). This study confirms that fermentation of microbial colony no. 59 gave rise to the production of antimicrobial compound(s) against antibiotic-resistant bacteria and that the antimicrobial compound(s) could be extracted using a hydrophobic resin.

ii) Flash Column Chromatography

A flash column chromatography was used to separate the antimicrobial compound(s) present in the concentrated resin eluates. Flash column (i.e., C-18 silica column) was prepared as followed.

Without being bound by a theory, it is believed that a flash column functions to separate a compound (such as the present antimicrobial compound) from other compounds present in a fermentation broth. It is further believed that the present antimicrobial compound can effectively be separated by a flash column when loaded with a mixture containing the hydrophobic resin-bound material. Suitable flash columns may be used which include, but not limited to, C18, C8 and CN.

Two hundred and fifty grams (250 grams) of C-18 silica resin was placed into a 2 L flash chromatography column (60 cm×7.5 cm) (Aldrich). The C-18 silica resin was thoroughly reconstituted by running ˜2 L of 100% acetonitrile by gravity. In order to pack the flash chromatography column, 1 L of 100% acetonitrile was applied to the column and forced through under 5 lb/in² gauge of nitrogen gas. The packed C-18 silica was equilibrated with 2 L of 30% acetonitrile under 5 lb/in² gauge of nitrogen gas.

Concentrated resin eluate was adjusted to 30% acetonitrile to form a mixture. The mixture was applied slowly with a pipette to the equilibrated C-18 silica column. The mixture was allowed to enter the C-18 silica column by gravity. The applied mixture was subjected to chromatography under isocratic conditions using 30% acetonitrile as a mobile phase. A total of ten (10) fractions of 200 mL were collected. The presence of antimicrobial activity in the fractions was determined using the antimicrobial assay against MRSA as described above.

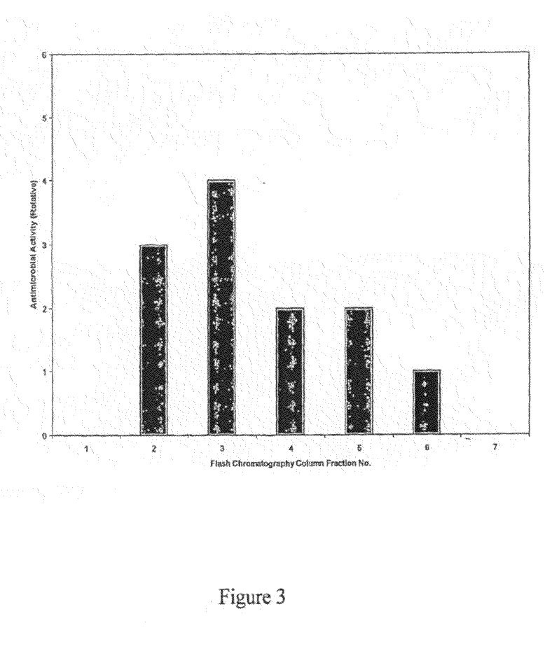

FIG. 3 shows that antimicrobial activity was strongly detected in fraction nos. 2, 3, 4, 5, and 6 while fractions 1 and 8 exhibited negligible antimicrobial activity. Fractions 2, 3, 4, 5 and 6 were pooled. The pooled fractions containing antimicrobial activity were concentrated 10-fold using a rotary evaporator. The concentrated pooled fractions were further subjected to two rounds of flash column chromatography. Antimicrobial activity was confirmed to be present in the pooled fractions without any diminution. This study confirms that extracted antimicrobial compound(s) bound onto a hydrophobic resin could be separated using a flash column chromatography.

iii) HPLC Chromatography

A HPLC chromatography was used to purify antimicrobial compound(s) present in the pooled fractions from flash column chromatography. A Shimadzu HPLC column containing an Econosil C-18 (22 mm×250 mm; 10μ particle size) was used. The HPLC column was equilibrated with 200 ml, of a solution containing 12% acetonitrile and 0.05% trifluoroacetic acid, at a flow rate of 9 mL/min.

Without being bound by a theory, it is believed that a HPLC column functions to bind to a compound (such as the present antimicrobial compound) so as to retain onto the HPLC column resin. It is further believed that the present antimicrobial compound can be effectively eluted using a gradient of acetonitrile and TFA. Suitable HPLC column resins may be used to retain the present antimicrobial compound via a hydrophobic-hydrophobic interaction. Exemplified HPLC column resins include, but not limited to, C18, C8 and CN.

The concentrated pooled fractions the flash column chromatography (−200 mL) was applied onto the equilibrated HPLC C-18 column. Elution was performed using a gradient of 18-30% acetonitrile+0.05% TFA over 15 minutes to remove the unbounded materials. Separation of the bound materials was performed using isocratic conditions of 30% acetonitrile+0.05% trifluoroacetic acid for 30 minutes. Approximately thirty (30) fractions (each fraction contains ˜6 mL) were collected. Antimicrobial activity present in the collected fractions (i.e., 20 fractions) was determined using antimicrobial assay against MRSA as described above.

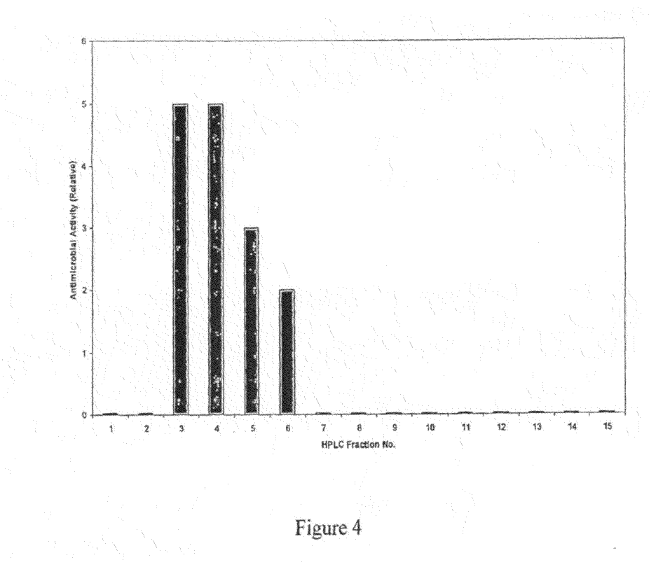

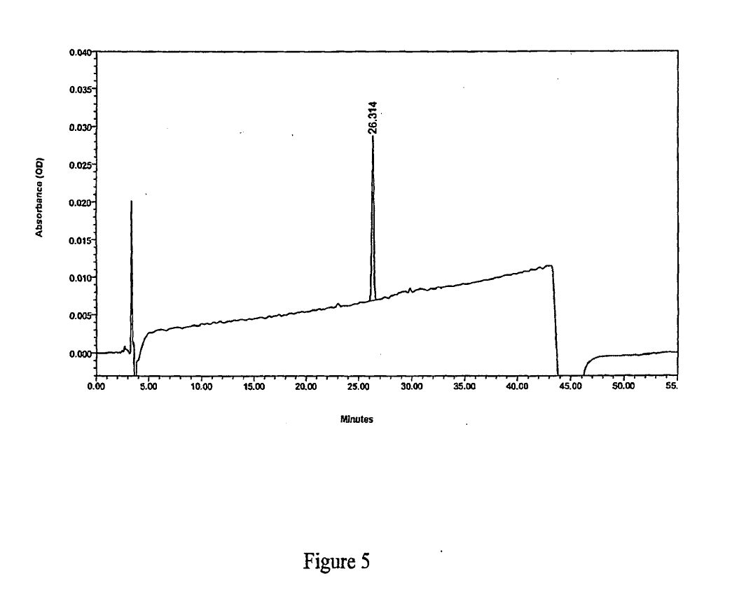

Out of the collected 30 fractions, four (4) fractions (fraction no. 3, 4, 5 and 6) exhibited the antimicrobial activity against MRSA. (See FIG. 4). Fraction no. 4, 5 and 6 contained a single peak that corresponded to the antimicrobial activity against MRSA. These two (2) fractions containing a single peak corresponding to the antimicrobial activity coincided with the HPLC peak having a retention time of ˜26.30 minutes. (See FIG. 5). The purity of antimicrobial compound was estimated to be >99% based on the HPLC analysis.

Several fractions from various experiments were pooled together and concentrated by rotary evaporation followed by lyophilization (i.e., freeze-dried). Approximately fifty (50) mg of the purified dried material were obtained. Noted that the 50 mg of the purified dried material was derived from 750 L of the fermentation broth after the fermentation culture. At a volume of 750 L, it is speculated that the antimicrobial compound is present at a concentration of about 60 μg/L in the fermentation broth. This study confirms that antimicrobial compound(s) separated from flash column chromatography can be purified to a homogeneity of >99% purity. An aliquot (i.e., 4 mg) of the purified dried material was subjected to NMR analysis for structural determination. Another aliquot (i.e., ˜0.5 mg) was used to measure the antimicrobial activity against a wide spectrum of microorganisms.

Structural Analysis of the Purified Antimicrobial Compound

In order to determine the structure of the HPLC purified antimicrobial compound isolated from fermentation broth of microbial colony No 59, NMR was performed on the HPLC purified peak material.

Nuclear Mallnetic Resonance (NMR) Analysis

After the compound was purified as described in the paragraphs above, the compound was analyzed by NMR spectroscopy to determine the structure. All of the NMR experiments were done on a Bruker NMR with a proton resonant frequency of 500 MHz (carbon 125 MHz). All NMR spectra were collected with the sample dissolved in deuterated methanol. For each experiment sufficient acquisitions were accumulated to provide an adequate signal to noise. NMR analysis was performed on the purified HPLC peak material recovered from the fermentation broth of microbial colony no. 59.

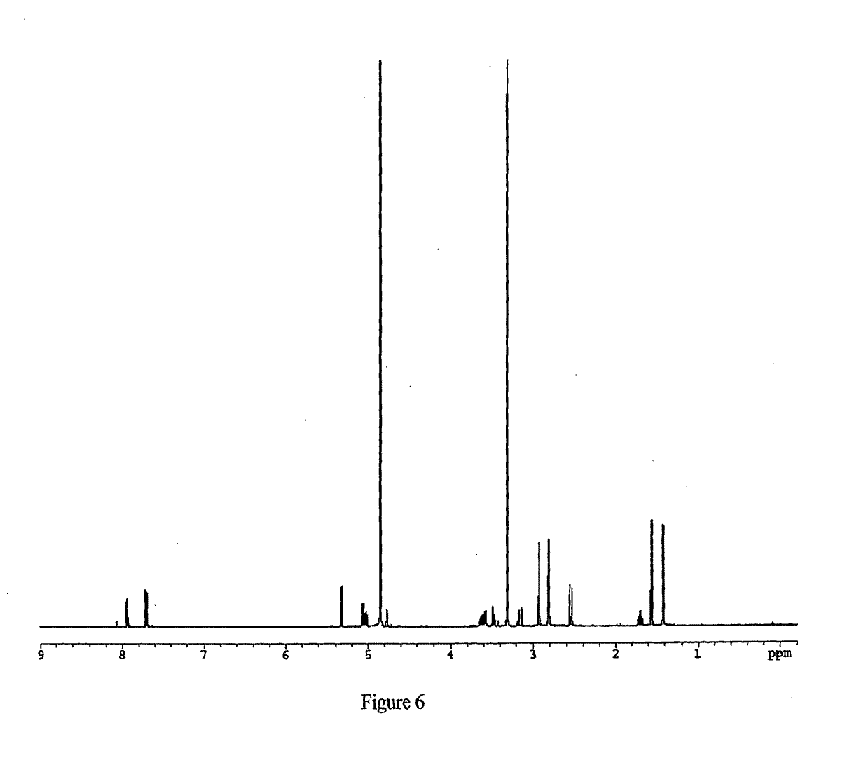

Firstly, 1D proton NMR experiment was performed, which provided the spectrum as indicate in FIG. 6. This experiment defines the individual protons and is consistent with the structure of the present invention (See FIG. 1).

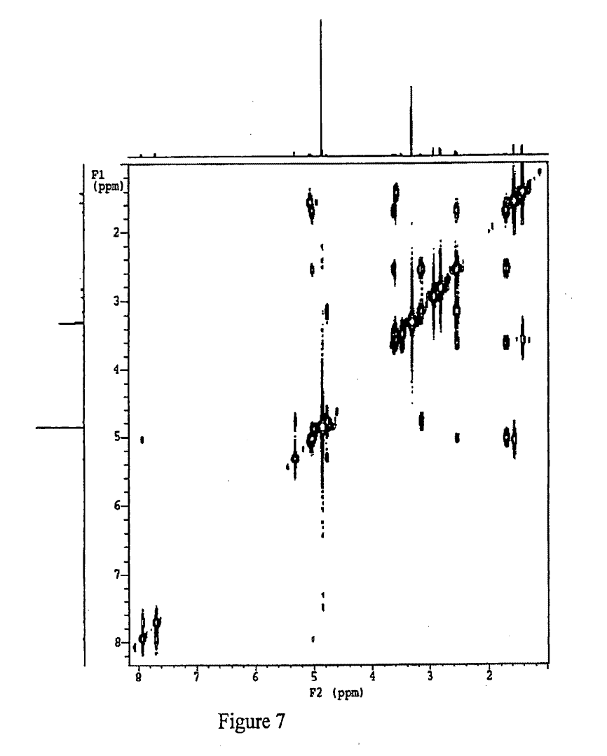

Secondly, 2D COSY experiment was performed, which provided the spectrum as indicated in FIG. 7. This experiment defines the position of each proton relative to the other protons and is consistent with the structure of the present invention (See FIG. 1).

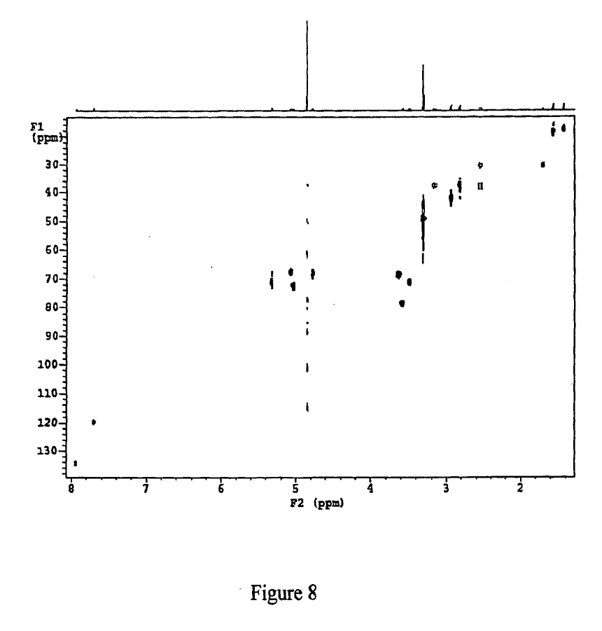

Thirdly, 2D TOCSY experiment was performed, which provided the spectrum as indicated in FIG. 8. This experiment defines the multiplicity of each carbon (CH₃, CH₂, CH) and is consistent with the structure of the present invention (See FIG. 1).

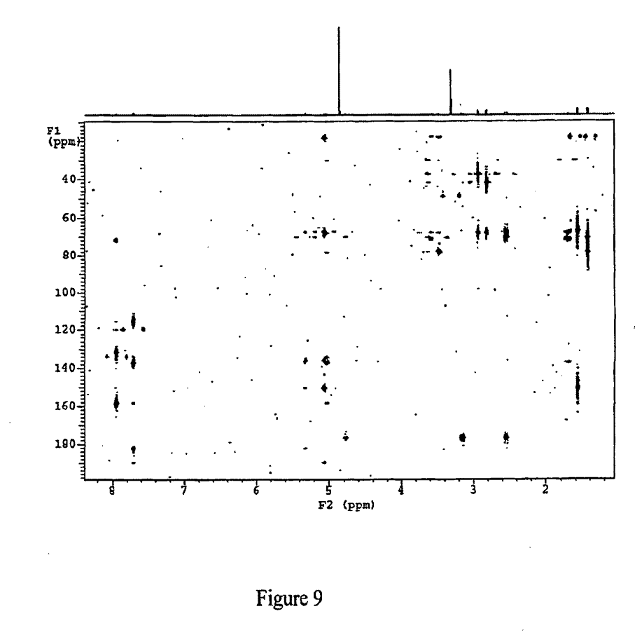

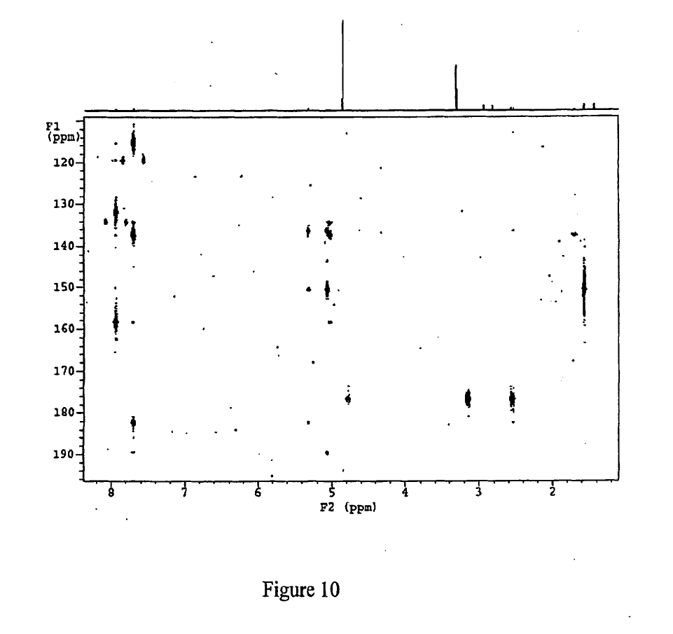

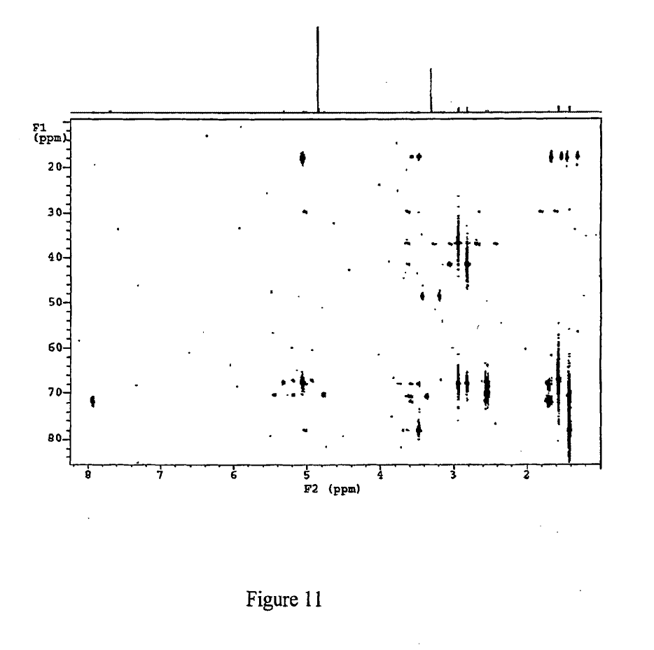

Fourthly, 2D HMBC experiment was performed, which provided the spectrum as indicated in FIG. 9, FIG. 10 and FIG. 11. This experiment defines the relationship of the carbons to each other and is consistent with the structure of the present invention (See FIG. 1).

The four spectra together conclusively show that the structure of the purified HPLC peak material is as presented in FIG. 1 of present invention. The results of the mass spectroscopy and NMR analysis identify the purified antimicrobial compound recovered from fermentation broth of microbial colony no. 59 as having a structural formula as depicted in FIG. 1. The antimicrobial compound has a chemical name of lactoquinomycin.

Lactoquinomycin A was first discovered in the mid 1970s and is referenced in U.S. Pat. No. 3,966,913 and Japanese Pat. No. 51061695, 54024479, 52015895 and 54027440. It has been shown to exert an antimicrobial activity against some gram-positive bacteria (such as Staphylococcus and Streptococcus) as well as against certain penicillin-resistant Staphylococcus. Lactoquinomycin exerted no antimicrobial activity against mycobacteria, (i.e. Mycobacteria smegmatis) (see Journal of Antibiotics 29(7), 756-8). Lactoquinimycin A also exerted no antimicrobial activity against methicillin or vancomycin resistant gram-positive bacteria. No pharmaceutical composition containing lactoquinomycin has been reported.

Purified Antimicrobial Compound of Lactoquinomycin: Evaluation of Antimicrobial Activity

Spectrum of the antimicrobial activities for the HPLC-purified antimicrobial compound, namely, lactoquinomycin (>99% purity) was further evaluated. The HPLC-purified material was diluted with sterile distilled water to a concentration of 0.1 μg/mL. The diluted HPLC-purified material was assayed for antimicrobial activity using the antimicrobial assay as described above. The diluted HPLC-purified material was assayed for its antimicrobial activity against ten (10) microorganisms; namely, 1) Staphylococcus aureus; 2) methicillin-resistant Staphylococcus aureus; 3) vancomycin-resistant Staphylococcus aureus; 4) Enterococcus faecilis; 5) vancomycin-resistant Enterococcus faecilis; 6) Mycobacterium smegmatis; 7) E. coli; 8) Pseudomonas aeriginosa; 9) Serratia marcesens; and 10) Candida albicans.

TABLE 3
Antimicrobial Spectrum of Activity for Purified Lactoquinomycin

| | Antimicrobial Activity For | Known Antibiotic |
|---|---|---|
| Tested Microorganisms | Purified Lactoquinomycin | Controls |
| Gram (+) Bacteria | | |
| Staphylococcus aureus | +++++ | vancomycin (++) |
| Methicillin-Resistant Staphylococcus aureus | +++++ | vancomycin (++) |
| Vancomycin-Resistant Staphylococcus | +++++ | (−)* |
| aureus | | |
| Enterococcus faecilis | +++ | vancomycin (++) |
| Vancomycin-Resistant Enterococcus faecilis | +++ | (−)* |
| Mycobacterium palustre | Not tested | vancomycin (++++) |
| Mycobacterium phlei | Not tested | vancomycin (++++) |
| Mycobacterium smegmatis | +++++ | vancomycin (++++) |
| Gram (+) Bacteria | | |
| E. coli | − | tetracycline (+++++) |
| Pseudomonas aeriginosa | − | gentamicin (+++++) |
| Serratia marcesens | − | gentamicin (+++++) |
| Fungi | | |
| Candida albicans | − | amphotericin B (+++++) |

“−” refers to “no detectable microbial activity”;
“+” refers to “weak antimicrobial activity”;
“+++” refers to “strong antimicrobial activity”; and
“+++++” refers to “very strong antimicrobial activity”; and
“*” refers to “no commercial antibiotic testing disc available”

As shown in the Table 3, the HPLC-purified antimicrobial compound lactoquinomycin exhibited strong antimicrobial activity against the tested gram-positive bacteria including methicillin and vancomycin-resistant Staphylococcus aureus, Enterococcus, and Mycobacteria. Notably, the HPLC-purified antimicrobial compound did not exhibit any antimicrobial effects against the tested gram-negative bacteria. The HPLC-purified antimicrobial compound also did not have any activity against the tested yeast and fungus, indicating specificity.

The present invention will be further illustrated by the following preferred examples, but should not be constructed as limited by those examples.

### Example 1

### Soil Preparation and Primary Screening

In separate experiments, 0.5 gram soil samples were diluted (20,000 fold) with distilled water. Aliquots of 100 μL, were inoculated onto twelve (12) agar plates as described above in order to perform primary screening for antimicrobial activity. The inoculated plates were allowed to incubate at 30° C. for two (2) weeks. The following microbial colonies were identified (in addition to the microbial colony no. 59) to exhibit a zone of inhibition against the neighboring colony on respective plates:
i) microbial colony nos. 25 and 26 (zone of inhibition on Czapek agar plate);
ii) microbial colony nos. 16 and 24 (zone of inhibition on Actinomycetes agar plate); and
iii) microbial colony no. 37 (zone of inhibition on Bennett agar plate).

These positive microbial colonies were subsequently streaked for isolation on fresh plates and glycerol stocks were prepared for long term storage.

### Example 2

### Secondary Screening

In separate experiments, secondary screening was performed on the microbial colony nos. 16, 24, 25, 26, and 37 (in addition to microbial colony no. 59). These microbial colonies were separately inoculated into respective seed culture media (2 mL) (i.e., microbial colony nos. 16 and 24 were cultured in the liquid version of Actinomycetes media; microbial colony nos. 25 and 26 were cultured in the liquid version of Czapek media; and microbial colony no. 37 was cultured in the liquid version of Bennett media).

The liquid cultures were allowed to incubate at 28° C. for 96 hours on a rotary shaker at 250 rpm. Aliquots (1 mL each) of culture broth were centrifuged (13,000 rpm; 2 minutes), dried in a SpeedVac, and resuspended in 100 μL of liquid culture media (10-fold concentrated). Antimicrobial activity using a paper disc assay was performed against Micrococcus luteus; E. coli; Candida albicans; and Pseudomonas aeriginosa as described above.

It was observed that microbial colony nos. 16 and 25 were active against Micrococcus luteus. Microbial colony no. 24 was active against all four (4) tested microorganisms. Microbial colony no. 26 was active against Candida albicans. Microbial colony no. 37 was active against Micrococcus luteus; E. coli; and Pseudomonas aeriginosa.

### Example 3

### Tertiary Screening

In separate experiments, tertiary screening was performed on the microbial colony nos. 16, 25, and 37 (in addition to microbial colony no. 59). These microbial colonies were separately inoculated into respective seed culture media (2 mL) (i.e., microbial colony no. 16 was cultured in the liquid version of Actinomycetes media; microbial colony no. 25 was cultured in the liquid version of Czapek media; and microbial colony no. 37 was cultured in the liquid version of Bennett media).

The liquid cultures were allowed to incubate at 28° C. for 96 hours on a rotary shaker at 250 rpm. Aliquots (1 mL each) of culture broth were centrifuged (13,000 rpm; 2 minutes), dried in a SpeedVac, and resuspended in 100 μL of liquid culture media (10-fold concentrated). Antimicrobial activity using a paper disc assay was performed against MRSA and Staphylococcus aureus as described above.

It was observed that microbial colony nos. 16 and 25 were active against MRSA and Staphylococcus aureus. Microbial colony no. 37 was active against Staphylococcus aureus.

### Example 4

### Fermentation of Microbial Colony No. 59

In this experiment, fermentation condition using a fermentor was evaluated. Seed culture medium (i.e., Emerson media) (2 mL) was inoculated with a loopful of an isolated colony of microbial colony no. 59. The inoculum was incubated at 28° C. for 48 hours on a rotary shaker at 250 rpm. Two (2) mL of the inoculum was transferred into 200 mL of Emerson media and allowed to incubate for an addition 8 hours at 28° C. on a rotary shaker at 250 rpm.

The 200 mL inoculum was added to 2 L of Emerson media in a 3.3 L BioFlo III fermentor and incubated at 28° C. for 9 days with mechanical stirring at a speed of 500 rpm and aeration (2 L/min). It was observed that mechanical stirring at a speed of 500 rpm during fermentation in the BioFlo III fermentor was similarly effective in the fermentation of the microbial colony no. 59 as compared to that of using a mechanical stirring speed of 250 rpm.

### Example 5

### Fermentation of Microbial Colony No. 59

In this experiment, fermentation condition using a shake flask was evaluated. Inoculation of microbial colony no. 59 in Emerson media (2 mL) was performed as in Example 4.

The 200 mL inoculum was added to 2 L of Emerson media in a 4 L Erlenmeyer flask and incubated at 28° C. for 9 days with rotary shaking at a speed of 350 rpm under ambient aeration.

It was observed that rotary shaking at a speed of 350 rpm during fermentation in the 4 L Erlenmeyer flask was similarly effective in the fermentation of the microbial colony no. 59 as compared to that of using rotary shaking at a speed of 250 rpm.

### Example 6

### Extraction of Fermentation Broth

In these series of experiments, the effects of different solvents on extraction of antimicrobial activity present in the fermentation broth were evaluated. Solvent extraction was performed after the step of fermentation broth preparation (see above). This solvent extraction represents another avenue to extract antimicrobial compound(s) from the fermentation broth in addition to the use of a hydrophobic resin, followed by flash column and HPLC. Two solvents (i.e., ethyl acetate and chloroform) were used for extraction.

i) Ethyl Acetate Extraction: Fermentation of microbial colony no. 59 and the preparation of a fermentation broth (1 L) for microbial colony no. 59 were prepared as described above.

Fermentation broth (1 L) was thoroughly mixed with ethyl acetate (1 L) in a Separatory funnel for about 3 minutes. The mixture was allowed to settle to form an upper organic phase (i.e., ethyl acetate) and a lower aqueous phase (i.e., fermentation broth). The upper organic phase (containing the antimicrobial activity) was collected. The lower aqueous phase was subjected to another cycle of extraction using ethyl acetate. The two (2) upper organic phases were combined and concentrated approximately ten-fold at room temperature and under vacuum. Antimicrobial activity of the upper organic phase was assayed against MRSA using a paper disc agar assay as described above. It was observed that ˜50% of the antimicrobial activity was extracted into the ethyl acetate. In contrast, the use of a hydrophobic resin can reach up to a ˜90% recovery of the antimicrobial activity from the fermentation broth.

ii) Chloroform Extraction: Fermentation of microbial colony no. 59 and the preparation of fermentation broth (1 L) for microbial colony no. 59 were prepared. The extraction procedure was performed as in ethyl acetate experiment except chloroform (1 L) was used. The chloroform mixture was allowed to settle to form an upper aqueous phase (i.e., fermentation broth) and a lower organic phase (i.e., chloroform). The lower organic phase (containing the antimicrobial activity) was collected. The upper aqueous phase was subjected to another cycle of extraction using chloroform. The two (2) lower organic phases were combined and concentrated approximately ten-fold at room temperature and under vacuum. Antimicrobial activity of the upper organic phase against MRSA was assayed using a paper disc agar assay as described above. It was observed that ˜50% of the antimicrobial activity was extracted into the chloroform. The use of a hydrophobic resin can reach up to a ˜90% recovery of the antimicrobial activity from the fermentation broth.

### Example 7

### Extraction of Fermentation Broth with Hydrophobic Resin

In these series of experiments, the effects of different eluants on hydrophobic resin were evaluated. Three (3) different eluants (i.e., methanol, acetone, and acetonitrile) were tested (in addition to the acetonitrile with TFA (0.05%) as described above).

i) Methanol Elution: Extraction of antimicrobial compound(s) from the prepared fermentation broth of microbial colony no. 59 was conducted as described before. The bound antimicrobial compound(s) were eluted from the hydrophobic resin (i.e., Diaion SP-207). The hydrophobic resin was mixed with 200 mL of a solution containing methanol (100%). The methanol resin mixture was incubated for 30 minutes at room temperature. The mixture was shaken (250 rpm) during the 30 minute-incubation time period. The mixture was filtered through a Buchner funnel to separate the hydrophobic resin. The separated hydrophobic resin was further eluted (2×) using 200 mL methanol (100%) and filtered. All eluants were combined and concentrated to 10-fold using a rotary evaporator. Antimicrobial activity of the concentrated eluants was tested against MRSA using a paper disc assay as described above. It was observed that ˜50% of antimicrobial activity was recovered using methanol as an eluant. This recovery is less than that observed using acetonitrile with TFA (0.05%) as an eluant.

ii) Acetone Elution: Fermentation broth and extraction using a hydrophobic resin were performed as in the above experiment, except acetone (100%) was used as an eluant. It was observed that ˜50% of antimicrobial activity was recovered using acetone as an eluant.

iii) Acetonitrile Elution: Fermentation broth and extraction using a hydrophobic resin were performed as in the above experiment, except acetonitrile (100%) was used as an eluant. It was observed that ˜50% of antimicrobial activity was recovered using acetonitrile as an eluant. These experiments indicate that elution of antimicrobial compound(s) using different solvents (e.g., methanol, acetone and acetonitrile) can be used.

Those skilled in the art will recognize, or be able to ascertain using no more than routine experimentation, many equivalents of the specific embodiments of the invention described herein. The disclosures of the cited publications in the present application are incorporated by reference herein in their entireties by reference. It is to be understood, however, that the scope of the present invention is not to be limited to the specific embodiments described above. The invention may be practiced other than as particularly described and still be within the scope of the accompanying claims.

What is claimed is:

1. A pharmaceutical composition comprising a compound having the formula of: 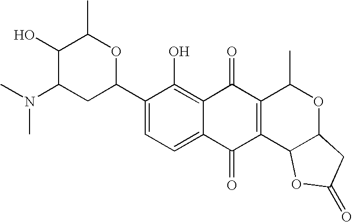
and a pharmaceutically acceptable excipient, wherein the pharmaceutical composition is effective for killing MRSA, VRSA, VRE and Mycobacteria.

2. The pharmaceutical composition according to claim 1, wherein the compound is a pharmaceutically acceptable salt thereof.

3. The pharmaceutical composition according to claim 1, wherein the compound is lactoquinomycin.

4. The pharmaceutical composition according to claim 1, wherein the compound is lactoquinomycin A.

5. The pharmaceutical composition according to claim 1, wherein the composition is a dosage form of a tablet or capsule.

6. The pharmaceutical composition according to claim 1, wherein the compound has a purity of greater than 90%.

7. The pharmaceutical composition according to claim 1, wherein the compound has a purity of greater than 95%.

8. The pharmaceutical composition according to claim 1, wherein the compound has a purity of greater than 99% purity.

9. A method of killing an antibiotic-resistant gram-positive bacterium selected from the group consisting of methicillin-resistant gram-positive bacterium and vancomycin-resistant gram-positive bacterium, comprising exposing the antibiotic-resistant gram-positive bacterium to an effective amount of a compound having the formula of: 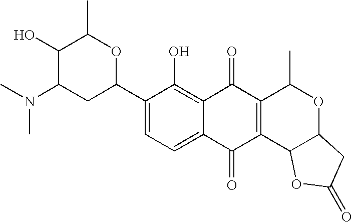
so as to kill said antibiotic-resistant gram-positive bacterium.

10. The method according to claim 9, wherein methicillin-resistant gram-positive bacterium is methicillin-resistant Staphylococcus aureus.

11. The method according to claim 9, wherein the methicillin-resistant gram-positive bacterium is methicillin-resistant Enterococcus faecilis.

12. The method according to claim 9, wherein the vancomycin-resistant gram-positive bacterium is vancomycin-resistant Staphylococcus aureus.

13. The method according to claim 9, wherein the vancomycin-resistant gram-positive bacterium is vancomycin-resistant Enterococcus faecilis.

14. A method of killing a mycobacterium, comprising exposing the mycobacterium to an effective amount of a compound having the formula of: 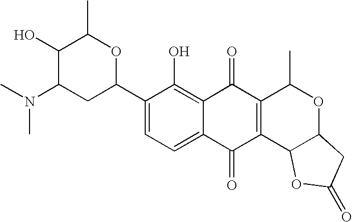
so as to kill said mycobacterium.

15. The method according to claim 14, wherein the mycobacterium is selected from the group consisting of Mycobacterium tuberculosis, Mycobacterium leprae, Mycobacterium avium complex, Mycobacterium avium subspecies paratuberculosis, Mycobacterium palustre, Mycobacterium phlei, and Mycobacterium smegmatis.

16. A method of treating an antibiotic-resistant microbial infection in a patient, comprising administering to a patient with a pharmaceutical composition containing a compound having the formula of:

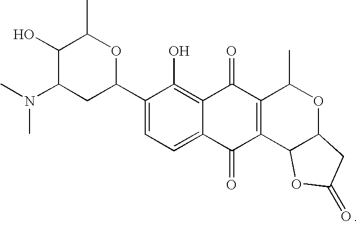

17. The method according to claim 16, wherein the antibiotic-resistant microbial infection is caused by Staphylococcus aureus or Enterococcus faecilis.

18. The method according to claim 16, wherein the microbial infection is a disease selected from the group consisting of bacteremia, pneumonia, osteomyelitis, cellulitis, abscesses, endocarditis, and urinary tract infection.

19. A method of treating a mycobacterial infection in a patient, comprising administering to a patient with a pharmaceutical composition containing an effective amount of a compound having the formula of:

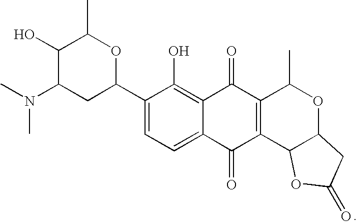

20. The method according to claim 19, wherein the mycobacterial infection is a disease selected from the group consisting of tuberculosis, leprosy, Mycobacterium avium complex associated disseminated disease in AIDS, and Mycobacterium avium subspecies paratuberculosis associated Crohn's disease.

21. A biologically pure culture of the microorganism, Streptomyces species (microbial colony no. 59, NRRL B-30919), said culture being capable of producing lactoquinomycin.

22. A fermentation broth obtained by fermenting the biological pure culture of claim 21 in a nutrient medium containing an assimilated source of carbon and nitrogen.

23. A fermentation process of preparing a compound having the formula of: 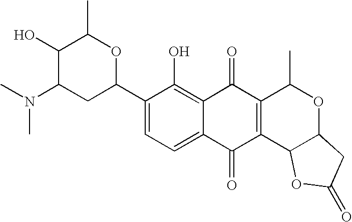
comprising the steps of:
a) culturing a microorganism, Streptomyces species (microbial colony 59, NRRL B-30919) in a fermentation medium; and
b) recovering said compound.

24. The process according to claim 23, wherein the compound is lactoquinomycin.

25. The process according to claim 23, wherein the culturing step is performed at a temperature of about 26° C. to about 40° C.

26. The process according to claim 23, wherein the culturing step is performed at a temperature of about 37° C.

27. The process according to claim 23, wherein the culturing step is performed at a pH of about 5 to about 9.

28. The process according to claim 23, wherein the culturing step is performed at a pH of about 5 to about 7.5.

29. The process according to claim 23, wherein the culturing step is performed for about 3 to about 12 days.

30. The process according to claim 23, wherein the culturing step is performed for about 9 days.

31. The process according to claim 23, wherein the recovering step is performed by:
a) extracting the compound from the fermentation medium with a hydrophobic resin;
b) separating the compound with a flash column; and
c) purifying the compound using a HPLC chromatography.

32. The process according to claim 31, wherein the purified compound has a purity of greater than 90%.

33. The process according to claim 31, wherein the purified compound has a purity of greater than 95%.

34. The process according claim 31, wherein the purified compound has a purity of greater than 99%.
